(19) United States
(12) Patent Application Publication
(10) Pub. No.: US 2012/0060836 A1
(43) Pub. Date: Mar. 15, 2012

(54) PATIENT CIRCUIT FOR IMPROVED SUPPORT DELIVERY

(76) Inventors: Michael EL-SHAMMAA, Anaheim, CA (US); Chris ZOLLINGER, Chino Hills, CA (US)

(21) Appl. No.: 12/879,908

(22) Filed: Sep. 10, 2010

(51) Int. Cl. A61M 16/00; A61F 7/12

(52) U.S. Cl. 128/204.17; 128/204.18; 128/205.23

(57) ABSTRACT

A patient circuit for delivering improved support to a patient. In one embodiment of the present technology, the patient circuit includes at least one limb that includes: an inlet; an outlet; an enclosing wall defining an interior flow passage between the inlet and the outlet; and a configurable region configured to be adjusted to alter a at least one modifiable characteristic of the at least one limb.

### FIELD OF THE INVENTION

The present technology relates generally to the respiratory field. More particularly, the present technology relates to a breathing circuit.

### BACKGROUND OF THE INVENTION

In general, a breathing circuit is an assembly of components which connects a patient's airway to a machine creating an artificial atmosphere, from and into which the patient breaths. For example, the machine may be a ventilator and the components may be a series of tubes. When the ventilator pushes air through a tube to a patient, the air is heated by a heating wire positioned within the tube. Different types of breathing circuits accommodate different flow rates of air. Sometimes during treatment, a caregiver needs to alter the breathing circuit to accommodate a different flow rate of air for the patient. In this situation, the caregiver replaces the breathing circuit being used with a different breathing circuit. When a breathing circuit is switched, a patient's breathing passageway is opened up, thereby exposing the patient to the risk of infections, such as ventilator associated pneumonia (VAP). Therefore, hospitals are concerned with procedures to try to limit the number of times that a caregiver has to open up a patient's breathing circuit.

### BRIEF DESCRIPTION OF THE DRAWINGS

FIG. 1 is a perspective view of a patient circuit, according to one embodiment of the present technology.

FIG. 2 is a perspective view at least one modifiable characteristic of a configurable region of a limb of a patient circuit, according to one embodiment of the present technology.

FIG. 3 is a perspective view of a patient circuit, according to one embodiment of the present technology.

FIG. 4 is a perspective view of a patient circuit, according to one embodiment of the present technology.

FIG. 5 is a flow diagram of a method of altering at least one modifiable characteristic of a limb of a patient circuit, according to one embodiment of the present technology.

The drawings referred to in this description should not be understood as being drawn to scale unless specifically noted.

### DETAILED DESCRIPTION OF THE INVENTION

The discussion will begin with an overview of the general use of breathing circuits and the limitations associated therewith. The discussion will then focus on embodiments of the present technology that provide a patient circuit for delivering improved support to a patient. The discussion will then focus on the method for using a patient circuit for delivering improved support to a patient.

### Overview

Breathing circuits are utilized to deliver such medical support as air and anesthetics from a machine that creates an artificial environment to a patient via tubes. Breathing circuits are used in surgical procedures. For example, in a most general case, breathing circuits comprise an inspiratory limb running from a ventilator to a patient and an expiratory limb running from the patient back to the ventilator. The ventilator pushes air through the inspiratory limb to reach the patient. The patient inhales this pushed air and exhales air into the expiratory limb.

If the air is cold when the patient inhales it, the patient's body works hard to try to warm up the air for ease of breathing. Thus, breathing circuits are designed with heating wires positioned within the interior of at least the inspiratory limb. If a heating wire is positioned within the inspiratory limb such that the heating wire stretches the full length of the inspiratory limb, then all of the air moving through the inspiratory limb becomes heated. Thus, the air arriving from the inspiratory limb into the patient's mouth is also well heated.

However, if the heating wire is positioned such that it stretches for only two thirds of the way towards the patient's mouth while within the inspiratory limb, then the heated air that travels the final section of the inspiratory limb to the patient's mouth has time to cool off. This is especially true if the flow rate of air being pushed by the ventilator is low. However, if the air being pushed by the ventilator is moving at a fast rate, then this air retains much of its heat since the cooling off time as the air moves through the unheated sections is relatively minimal.

If the heating wire within the inspiratory limb is positioned too close to the patient's mouth while the air is moving at a fast rate, then the heated air may be too hot and scorch the patient during inhalation. On the other hand, if the heating wire within the inspiratory limb is positioned too far away from the patient's mouth while the air is moving at a slow rate, then the air may be too cool for the patient, thereby causing the patient's body to work harder to heat up the air.

Thus, a variety of breathing circuits exist that account for providing a desired temperature of heated air to the patient while also applying a desired rate of air flow to the patient. If it is determined that a patient needs a high rate of air flow during treatment, but is currently using a breathing circuit that only accommodates a low rate of air flow, the caregiver must disengage the breathing circuit from the patient and replace it with an appropriate breathing circuit. For example, the caregiver may decide that there is a need to increase the flow rate of air to 5 L/min from 2 L/min. The caregiver would change the breathing circuit to one that has a heating wire that is positioned to be farther from the patient.

Replacing breathing circuits creates a risk for the patient. Every time that a patient's breathing airway is opened up, the patient is exposed to the risk of infections, such as ventilator associated pneumonia (VAP). Furthermore, the hospital must stock a variety of breathing circuits to accommodate patients' breathing needs during surgical treatment. More particularly, a hospital may have to stock a large number of different breathing circuits in advance of performing services for the patient. Thus, the current method of exchanging one breathing circuit for another during treatment creates a health risk for the patient and is costly for the caregiver to accommodate.

Embodiments of the present technology provide a system for adjusting a position of a wire within a breathing circuit to accommodate a different flow rate of air. While the following discussion will address the flow rate of air, anesthetics, etc. For example, a portion of an inspiratory limb may be expanded such that the wire positioned within the limb is positioned further away from the patient than its original position. In another example, a portion of the inspiratory limb may be compressed such that the wire positioned within the limb is positioned closer to the patient then its original position. Thus, during medical treatment, the same breathing circuit components may be used for both low and high rates of air flow. By using embodiments of the present technology, caregivers will not have to open up a patient's airway multiple times, thus exposing the patient to bacteria causing infections. Further, in anticipation of providing medical services to patients, hospitals will only have to stock one type of breathing circuit instead of multiple types, thus saving money and other resources.

The following discussion will begin with a description of the structure of the components of the present technology. This discussion will then be followed by a description of the components in operation.

### Structure

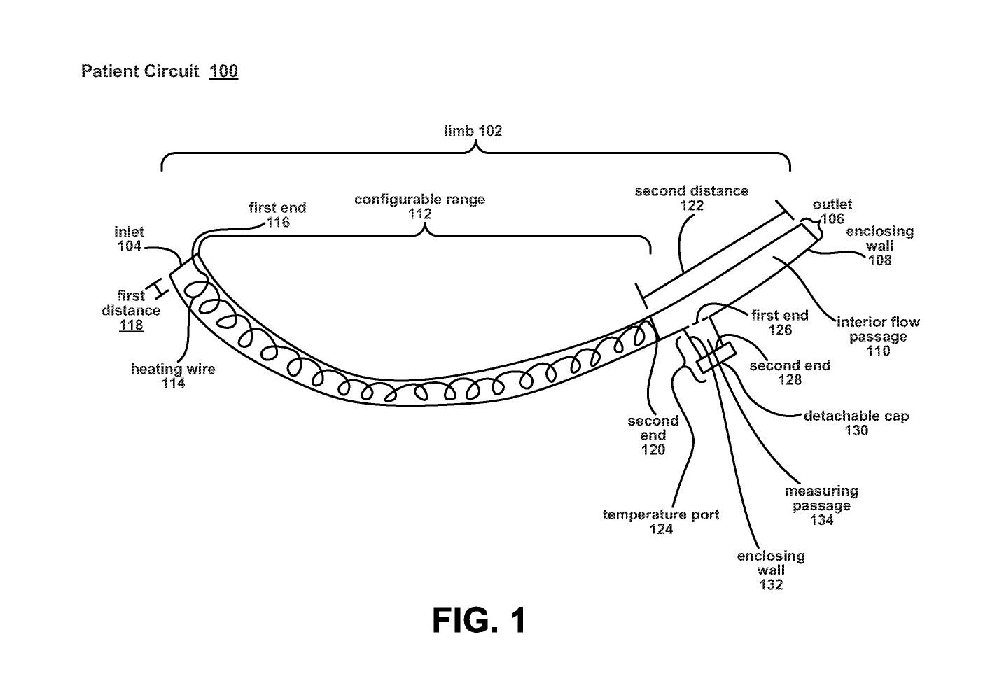

With reference now FIG. 1, a perspective view of a patient circuit is 100 is shown for delivering improved support to a patient. In this embodiment, the patient circuit 100 includes at least one limb 102. The at least one limb 102 refers to one or more limbs comprising the patient circuit 100. However, for purposes of brevity and clarity, reference to the at least one limb 102 will be made in the singular. Limb 102 includes an inlet 104, an outlet 106, an enclosing wall 108 defining an interior flow passage 110 between the inlet 104 and the outlet 106, and a configurable region 112 configured to be adjusted to alter at least one modifiable characteristic of the limb 102.

In one embodiment, configurable region 112 comprises corrugated material, such that the configurable region 112 may be expanded and compressed by being pulled and pushed. More particularly, the configurable region 112 may be made of any material that allows the compression and expansion therefore and is safe for use in a surgical procedure. In embodiments of the present technology, the shape of the configurable region 112 may also be changed. The at least one modifiable characteristic of the limb 102 includes the expandability of at least one portion of the configurable region 112. In another embodiment, the at least one modifiable characteristic of the limb 102 includes the compressibility of at least one portion of the configurable region 112. The portion that gets expanded or compressed may be the whole of the configurable region 112 or one or more separate portions of the configurable region 112.

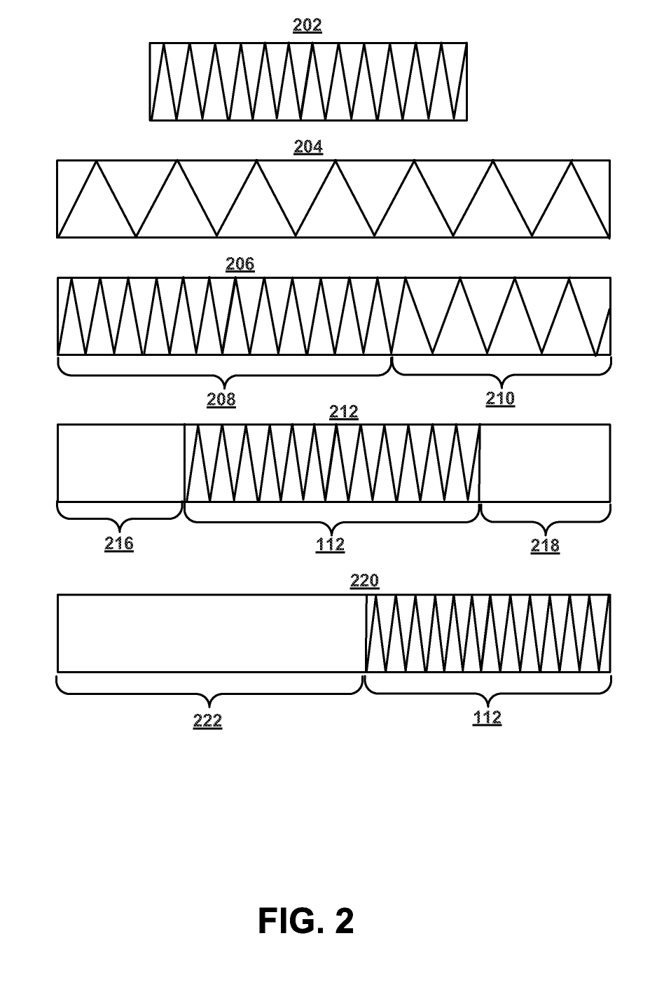

Referring now to FIG. 2, examples of, but not limited to, a configuration region 112 in various states of expansion and compression. For example, and referring to 202, a configurable region in a compressed state is shown. Referring now to 204, the configurable region of 202 is shown in an expanded state. Referring now to 206, a configurable region in a partially compressed 208 and partially expanded 210 state is shown. Referring now to 212, a configurable region 112 sandwiched between two nonconfigurable regions 216 and 218 is shown. Referring now to 220, a configurable region 112 is shown coupled with a nonconfigurable region 222. Thus, as can be seen, the limb 102 may be designed to comprise an assortment of configurations involving the configurable region 112.

In one embodiment, the patient circuit 100 further includes at least one heating wire 114 positioned within the interior flow passage 110 of the limb 102. The heating wire 114 is configured for heating the interior flow passage 110 when activated. Activation occurs when an active heating source is connected with a heating wire 114. In one embodiment, the heating wire 114 includes a first end 116 positioned a first distance 118 from the inlet 104 and a second end 120 positioned a second distance 122 from the outlet 106. A second distance 122 that is a small value means that the second end 120 of the heating wire 114 is positioned close to the patient, compared with a larger value. A second distance 122 that is a large value means that the second end 120 of the heating wire 114 is positioned farther away from the patient. Thus, in one embodiment, the first and second distances, 118 and 120, respectively, are variable and dependent upon a state of the at least one modifiable characteristic of the limb 102.

Embodiments of the present technology comprise a heating wire 114 of varying shapes. For example, in one embodiment, the heating wire 114 is helically wound. In another embodiment, portions of the heating wire 114 are helically wound and other portions of the heating wire 114 are non-helically wound. In still other embodiments, the heating wire 114 is non-helically wound.

Additionally, and referring briefly to FIG. 2, the heating wire 114 may reside in just a portion less than a whole of the configurable region or reside along the whole length of the configurable region. Thus, a configurable region may have heated and non-heated sections.

In one embodiment, the patient circuit 100 comprises a temperature port 124 that is coupled with the limb 102. The temperature port 124 has a first end 126 open to the interior flow passage 110, a second end 128 detachably sealed with a detachable cap 130 and an enclosing wall 132 defining a measuring passage 134 between the first end 126 and the second end 128. The measuring passage 134 is configured for receiving a temperature measuring device for measuring a temperature of the interior flow passage 110. For example, the temperature measuring device is placed within the temperature port 124 such that a portion of the temperature measuring device falls within the interior flow passage 110. The end of the temperature measuring device meets the center of the interior flow passage 110. Thus, the temperature measuring device is able to determine the temperature of the air that flows by it within the interior flow passage 110.

In one embodiment, the temperature port 124 is positioned to be open to the configurable region 112. In another embodiment, the temperature port 124 is positioned in a non-heated section of the limb 102. In another embodiment, the temperature port 124 is positioned in a non-heated section of the limb 102, which is directly adjacent to the configurable region 112. In other words, the temperature port 124 may be positioned between the configurable region 112 that is heated and a nonheated section that couples with the patient.

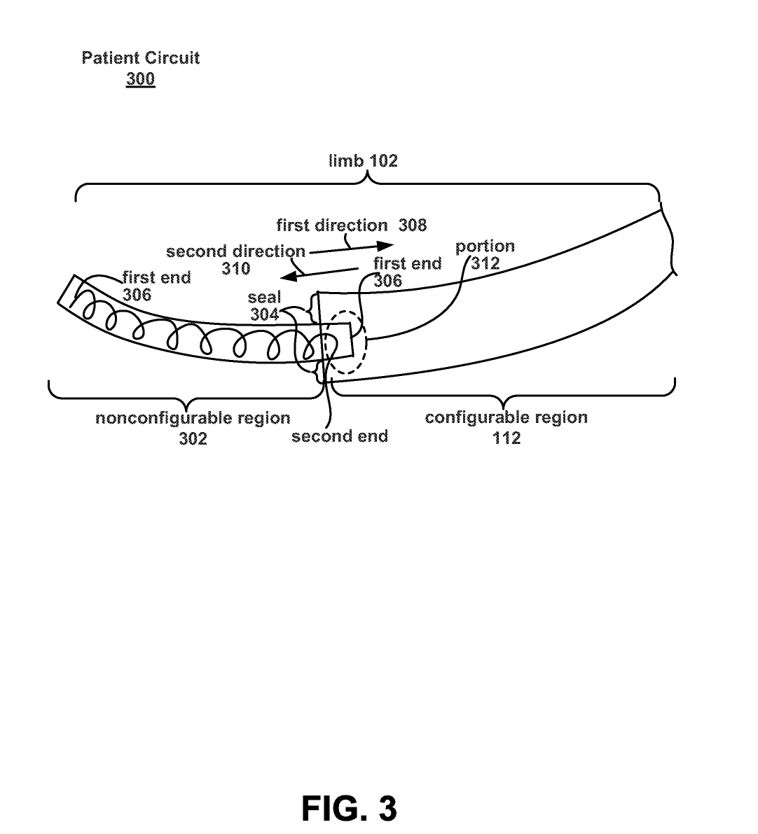

Referring now to FIG. 3, a perspective view of a patient circuit 300 is shown in accordance with an embodiment of the present technology. In one embodiment, patient circuit 300 includes a nonconfigurable region 302. In one embodiment, the configurable region 112 sealingly engages the nonconfigurable region 302 while a first end 306 of the configurable region 112 receives in a first direction 308 a sliding portion 312 of the nonconfigurable region 302. By sealingly engages, it is meant that between the nonconfigurable region 302 and the configurable region 112, a seal is provided such that particles may not travel through the seal. Patient circuit 300 shows a type of telescoping tubing involving configurable regions as described herein. Thus, while the nonconfigurable region 302 may be slid in and out of the configurable region 112, portion of the configurable region 112 itself may be expanded and compressed via its corrugated structure.

Referring now to FIGS. 1 and 3, in one embodiment, the limb 102 comprises a color coding configured for providing instructions for adjusting the configurable region 112, thereby modifying the at least one modifiable characteristic. For example, a portion of the limb 102 may have colors thereon, that when the limb 102 is compressed, colors are not seen, and when the limb 102 is expanded, the colors are seen. Instructions may be give to the caregiver to push or pull the configurable region 112 until a specific color is seen or no longer seen. The portion may be that of the configurable region 112, the nonconfigurable region 302, or combination thereof.

In one embodiment, a portion of the limb 102 may have a written indicator positioned thereon. In this case, the caregiver may need only to read the written indicator to determine how much to push or pull the configurable region 112. In another embodiment, the patient circuit 100 includes a lock holding the configurable region 112 in place upon activation of the lock. The lock may be activated by the caregiver. This lock prevents the accidental compression of the configurable region 112.

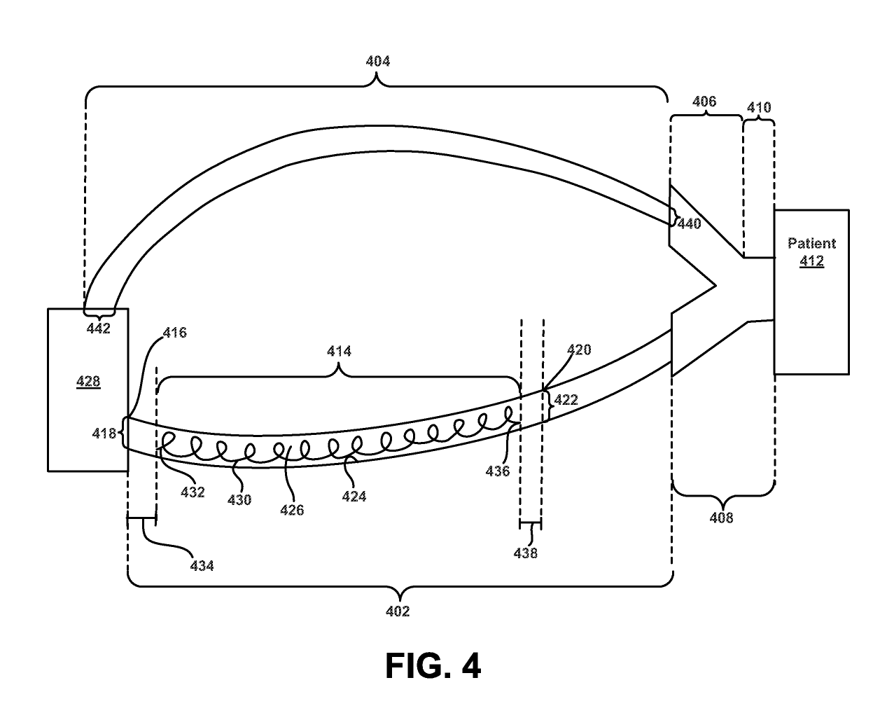

In one embodiment and referring to FIGS. 1 and 4, a perspective view of a patient circuit 400, is shown according to an embodiment of the present technology. While incorporating the features of the patient circuit 100 of FIG. 1, a further embodiment includes first limb 102, a second limb 104 coupled with the first limb 402 via a first connector end 406 of a limb connector 408, and a second connector end 410 of the limb connector 408 coupled with a patient 412.

Referring still to FIG. 4, a configurable region 414 of the limb 102 of a patient circuit 400 for altering modifiable characteristics of the limb 102 is shown in accordance with embodiments of the present technology. Of note, the limb 102 may be an inspiratory limb. Additionally, and as shown in FIG. 4, in one embodiment another limb 104 is an expiratory limb. In one embodiment, the configurable region 414 includes a first end 416 comprising an inlet 418, a second end 420 comprising an outlet 422. Further, configurable region 414 includes an adjustable enclosing wall 424 defining an interior flow passage 426 between the first end 416 and the second end 420, wherein the adjustable enclosing wall 424 is configured for being adjusted, thus altering a modifiable characteristic of the limb 102.

In one embodiment, the configurable region 414 includes a first end 416 coupled with a ventilator 428. In one embodiment, the configurable region 414 includes at least one heating wire 430 positioned within the interior flow passage 426 when activated. The at least one heating wire 430 comprises a first end 432 positioned a first distance 434 from the inlet 418 and a second end 436 positioned a second distance 438 from the outlet 422.

In one embodiment, the modifiable characteristic of the limb 102 comprises a length of the configurable region 414. In other words, the length of the configurable region 414 may be expanded and compressed.

As stated herein, in one embodiment, the configurable region 414 may include instructions for modifying the modifiable characteristics. For example, the configurable region 414 may have positioned thereon a color coding and/or a written indicator. Further, and as described herein, the configurable region 414 may include a lock holding the configurable region 414 in place upon activation of the lock.

Referring still to FIG. 4, in one embodiment, the patient circuit 400 is a breathing circuit for delivering respiratory support to the patient 412. Thus, in one embodiment, the breathing circuit comprises a limb 102 that is an inspiratory limb. The limb 402 includes an inlet 418 coupled with a ventilator 428, an outlet 422 coupled with the patient 412, an enclosing wall defining an interior flow passage 426 between the inlet 418 and the outlet 422. Further, embodiments include a configurable region 414 to be adjusted to alter a length of the limb 402. Embodiments further include at least one heating wire 430 positioned within the interior flow passage 426 when activated, wherein the at least one heating wire 430 comprises a first end 432 positioned a first distance 434 from the inlet 418 and a second end 436 positioned a second distance 438 from the outlet 422.

In one embodiment, the breathing circuit 400 includes a limb 104 that is an expiratory limb and that includes an inlet 440 coupled with the patient 412 and an outlet 442 coupled with the ventilator 428.

Thus, embodiments of the present technology provide for a single breathing circuit to deliver air, anesthetics, etc. to a patient, regardless of the airflow rate that is required. The present embodiments of the breathing circuit have a significant positive impact on the health of a patient.

### Operation

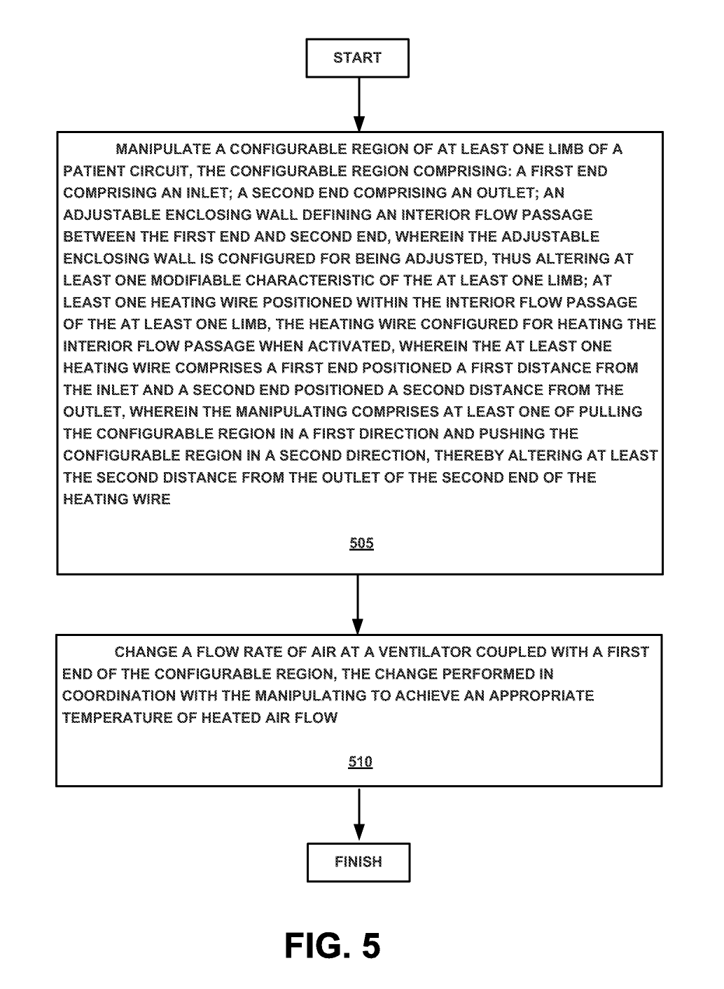

Referring now to FIG. 5, a flow diagram of a method of altering at least one modifiable characteristic of a limb of a patient circuit, is shown in accordance with one embodiment of the present technology. Referring now to 505 of FIG. 5 and to FIG. 4, a configurable region 414 of at least one limb 402 of a patient circuit 100 is manipulated, wherein the manipulating comprises at least one of pulling the configurable region 414 in a first direction and pushing said configurable region in a second direction, thereby altering at least a second distance 438 from the outlet 422 of the second end 436 of the heating wire 430.

In one embodiment, the method of altering at least one modifiable characteristic of limb 402 of the patient circuit 400 includes changing 510 a flow rate of air at a ventilator 428 coupled with a first end 416 of the configurable region 414. The changing is performed in coordination with the manipulating to achieve an appropriate temperature of heated airflow.

All statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A patient circuit for delivering improved support to a patient, said patient circuit comprising:
at least one limb comprising:
an inlet;
an outlet;
an enclosing wall defining an interior flow passage between said inlet and said outlet; and
a configurable region configured to be adjusted to alter at least one modifiable characteristic of said at least one limb.

2. The patient circuit of claim 1, further comprising:
at least one heating wire positioned within said interior flow passage of said at least one limb, said heating wire configured for heating said interior flow passage when activated, wherein said at least one heating wire comprises a first end positioned a first distance from said inlet and a second end positioned a second distance from said outlet.

3. The patient circuit of claim 2, wherein said at least one heating wire is helically wound.

4. The patient circuit of claim 1, wherein said at least one modifiable characteristic comprises an expandability of at least one portion of said configurable region.

5. The patient circuit of claim 1, wherein said at least one modifiable characteristic comprises a compressibility of at least one portion of said configurable region.

6. The patient circuit of claim 2, wherein said first and second distances are variable and dependent upon on a state of said at least one modifiable characteristic of said at least one limb.

7. The patient circuit of claim 1, further comprising:
a temperature port coupled with said at least one limb, said temperature port having a first end open to said interior flow passage, a second end sealed with a detachable cap and an enclosing wall defining a measuring passage between said first end and said second end, said measuring passage configured for receiving a temperature measuring device for measuring a temperature of said interior flow passage.

8. The patient circuit of claim 1, further comprising:
a nonconfigurable region, wherein said configurable region sealingly engages said nonconfigurable region while a first end of said configurable region receives in a first direction a sliding portion of said nonconfigurable region.

9. The patient circuit of claim 1, wherein said at least one limb further comprising:
a color coding configured for providing instructions for adjusting said configurable region, thereby modifying said at least one modifiable characteristic.

10. The patient circuit of claim 1, wherein said at least one limb comprises:
a first limb;
a second limb coupled with said first limb via a first connector end of a limb connector; and
a second connector end of said limb connector coupled with said patient.

11. A configurable region of at least one limb of a patient circuit for altering at least one modifiable characteristics of said at least one limb, said configurable region comprising:
a first end comprising an inlet;
a second end comprising an outlet; and
an adjustable enclosing wall defining an interior flow passage between said first end and said second end, wherein said adjustable enclosing wall is configured for being adjusted, thus altering an at least one modifiable characteristic of said at least one limb.

12. The configurable region of claim 11, wherein said first end is coupled with a ventilator.

13. The configurable region of claim 11, further comprising:
at least one heating wire positioned within said interior flow passage of said at least one limb, said heating wire configured for heating said interior flow passage when activated, wherein said at least one heating wire comprises a first end positioned a first distance from said inlet and a second end positioned a second distance from said outlet.

14. The configurable region of claim 11, wherein said at least one modifiable characteristic of said at least one limb comprises a length of said configurable region.

15. The configurable region of claim 11, further comprising instructions for adjusting said configurable region, thus modifying said at least one modifiable characteristic.

16. The configurable region of claim 15, further comprising:
a color coding positioned on said at least one limb.

17. The configurable region of claim 15, further comprising:
a written indicator positioned on said at least one limb.

18. The configurable region of claim 15, further comprising:
a lock holding said configurable region in place upon activation of said lock.

19. A method for using a patient circuit for delivering improved support to a patient, said method comprising:
manipulating a configurable region of at least one limb of a patient circuit, said configurable region comprising:
a first end comprising an inlet;
a second end comprising an outlet;
an adjustable enclosing wall defining an interior flow passage between said first end and said second end, wherein said adjustable enclosing wall is configured for being adjusted, thus altering an at least one modifiable characteristic of said at least one limb;
at least one heating wire positioned within said interior flow passage of said at least one limb, said heating wire configured for heating said interior flow passage when activated, wherein said at least one heating wire comprises a first end positioned a first distance from said inlet and a second end positioned a second distance from said outlet,
wherein said manipulating comprises at least one of pulling said configurable region in a first direction and pushing said configurable region in a second direction, thereby altering at least said second distance from said outlet of said second end of said heating wire.

20. The method of claim 19, further comprising:
changing a flow rate of air at a ventilator coupled with a first end of said configurable region, said changing performed in coordination with said manipulating to achieve an appropriate temperature of heated airflow.
